(19) United States
(12) Patent Application Publication
(10) Pub. No.: US 2011/0086872 A1
(43) Pub. Date: Apr. 14, 2011

(54) Berberine as a selective lung cancer agent and other compositions and methods

(76) Inventor: Maung Tin-Wa, San Francisco, CA (US)

(21) Appl. No.: 12/806,886

(22) Filed: Aug. 23, 2010

Related U.S. Application Data
(63) Continuation of application No. 11/881,425, filed on Jul. 26, 2007, now abandoned.

(51) Int. Cl. A61K 31/4745; A61P 35/00; C12N 5/09

(52) U.S. Cl. 514/280; 435/366

(57) ABSTRACT

Berberine or its salts or derivatives are identified as the active compound for selectively inhibiting lung cancer, potentially without toxic side effects. Berberine is preferably obtained by synthesis or partial synthesis, or is obtained from natural sources, such as Coptis teeta, or other berberine containing plants. Berberine and its derivatives are also active against HIV, and may be a safe new drug for the prevention of AIDS, alone or in combination with other antiviral agents. Composition and method of inhibiting tumor or viral infections and malaria without toxic side effects. A natural composition from the rhizome of Coptis teeta may be used as a safe new drug for the prevention of human breast cancer.

### CROSS-REFERENCE TO RELATED APPLICATIONS

This is a continuation application of prior filed U.S. divisional application Ser. No. 11/881,425 filed on Jul. 26, 2007, which application claims the benefit of U.S. application Ser. No. 10/429,104 filed on May 1, 2003, which application claims the benefit of U.S. provisional application Ser. No. 60/377,388 filed on May 3, 2002.

### STATEMENT REGARDING FEDERALLY SPONSORED RESEARCH

Not applicable.

### BACKGROUND OF THE INVENTION

Berberine is an alkaloid present in various species of Berberis and several other plant families. Oral berberine has both antisecretory and antimicrobial properties and is nontoxic at high doses.

Traditional healers in Myanmar, India and China have used crude berberine extracts from plants for many centuries in managing a variety of medical conditions, including acute diarrhea. In the laboratory, berberine shows in vitro activity against the protozoa Trichomonas vaginitis, Giardia lambia, Entamoeba histolytica, several of the protozoa strains, which cause leishmaniasis, as well as several types of fungi and bacteria. Berberine, purified as hydrochloride, sulfate, or tannate salts, has been used to treat bacterial, fungal and some protozoa in clinical studies. Orally administered berberine has been shown to be a safe and effective agent against diarrhea, such as that caused by the protozoal pathogen G. lambia. Healing of sores caused by cutaneous species of leishmanial parasites has been effected by intradermal administration of berberine. Berberine has also proven useful in the treatment of acute diarrhea induced by Escherichia coli and Vibrio cholerate toxins.

Other uses of various berberine are disclosed in Maroko, U.S. Pat. Nos., 5,153,178; 4,980,344; 4,749,708; and 4,761,417; each of which is incorporated herein by reference. These patents include many references on berberine and protoberberine type compounds, from synthesis, biological activities, pharmacology, pharmacokinetics, clinical trials, etc.

In U.S. Pat. No. 5,876,728, Kass discloses a composition containing three herbs or seven herbs for use in treating a variety of cancers. The idea of using safe nontoxic herbs, a few of which showed activity against a few individual cancers is a step in the right direction. However, the ‘shot-gun’ approach expecting to cure all types of cancer appeared far too optimistic and hardly useful. It is more of a hit or miss method, without selectivity toward many different forms of cancer, and cannot be fruitful. It is not making use of recent advances in molecular biology and natural products chemistry in a meaningful way.

In U.S. Pat. No. 6,290,995, Xinxian offered, in Example 3, a new radioimmunoassay (RIA) method for precise determination of berberine and bacailin, but not other compounds present in the drug product. This RIA method will serve a useful purpose for large clinical program including double blind analysis and good clinical practice. The selection of only a combination of two crude drugs and utilizing the current advances of biology is an improvement over the Kass patent (U.S. Pat. No. 5,876,728) mentioned above. However, new technology in natural products was ignored. The product obtained by the processes of years past is unfortunate.

Even though Xinxian claimed that the two products were berberine and bacailin, the method used for producing them (Examples 1 & 2) would not have provided pure bacailin or pure berberine. The final product in each case was crude extract/fraction containing bacailin and other components, and a crude extract/fraction containing berberine and other components, and should have been identified as such—not as bacailin and berberine as though they were pure compounds. A person knowledgeable in natural products chemistry would know there might be other components in the “crude” extract/fraction described.

These components (or “impurities”) in admixture with bacailin and/or berberine could very well be eliciting the nonselective activities in the different cancer cell lines. Thus, all the results of the biological testing (Examples 5 to 10) are inconclusive as to the real activities of pure bacailin and/or pure berberine. Activities in some cancer cell lines may be due to these “impurities” and may not be due to pure berberine. The disclosure relates generally to some observed cytotoxic properties of the mixture of two crude drug products (containing other compounds besides berberine) against several cancer cell lines. It does not relate to the specific activity of pure berberine against specific conditions—namely lung cancer.

Xinxian listed several plant sources. From a chemotaxonomic point of view these are expected to contain berberine, however, those knowledgeable in the art understand that different species of the same genus contain different amounts of the same substances (e.g. berberine) along with other components of varying amounts. Alkaloids and/or other plant constituents vary considerably depending upon the origin of the plant. (See “The Isoquinoline Alkaloids, Chemistry and Pharmacology, Shamma, Academic Press, New York 1972; Chapter 10 and the Chemistry of Isoquinoline Alkaloids, Tetsuji Kametani, Elsiver Publishing Co., New York, 1996).

Thus, while Xinxian describes biological activities, the method of selection of plants and the production of the final product does not lead to pure berberine. Any conclusions reached regarding the non-selective cytotoxic activities of berberine may be misleading.

Similar to Xinxian's patent, Han et al. (U.S. Pat. No. 5,928,645 of extracted substance having anti-HIV activity) selected only two plant extract/fractions. It consists of a mixture of a non-fat starch from Ricinus communis semen and a root of Coptis species, the latter of which was not fully identified botanically (no species name given). Hence, the disadvantages inherent in Xinxian and Kass apply. Also, since Han did not scientifically identify the species of Coptis used, quality control of the product produced by Han's invention also would be a great problem, as explained previously—alkaloids and/or other plant constituents vary considerably depending upon the origin of the plant” as stated in the references above (Shamma, 1972 and Tetsuji, 1996).

The Han method used obtain the composition of the extracted substance having anti-HIV is long and tedious and would not be commercially feasible. Even though the state of the art technology in both biology and analytical chemistry (such as UV, HPLC, NMR, IR etc.) were employed (FIGS. 1 through 22), the outcome is nonetheless no better than the results of Kass and Xinxian, as they will not be reproducible because of the varying compositions. Furthermore, the experiments described (e.g., FIG. 3 through 5 and tables 1 to 3, Examples 1 and 7) did not provide important details, such as, the weight of samples or volumes of solvents used.

Tan et al., Biochem Biophysics. Res. Commun. 185: 370-378 (1992) indicated berberine and a few other protoberberines to exhibit HIV-1 and HIV-2 reverse transcriptase inhibitions.

Dai et al., Phytother. Res. 7:290-294 (1993) reported that berberine showed cytotoxicity against various human cancer cell lines: Colon, Col 2 (IC 50=40.0 uM); Breast, BC1 (2.74 uM); and Lung, Lu1 (25.0 uM). In other words, Dai's work concluded that berberine was not selective. The berberine isolated by Dai may not be pure and may contain other compounds as impurities that may be responsible for the activities against other human cell lines besides lung cancer cells. A similar conclusion was arrived at by Xinxian in his 2001 patent disclosure, i.e., berberine was nonselective, exhibiting activities in different cancer cell lines, probably as a result of crude extract or fraction of berberine obtained.

The present invention overcomes one or more deficiencies of the related art. This was accomplished by utilizing only one plant that is properly and scientifically identified in contrast to Han (U.S. Pat. No. 5,928,645) where the identification of one of the two plants used was incomplete and Xinxian (U.S. Pat. No. 6,290,995) where two crude plants were used.

The extraction method described also involves fewer steps to concentrate the biological activities in a few fractions, each having specific activity against lung cancer, breast cancer, HIV and malaria. Such quality controlled compositions involving stepwise extraction procedures offer considerable advantages both in terms of product quality and commercial applications. Because the biological activities are localized/isolated in specific fractions, isolation of active compound(s) is straightforward and targeted. This is clearly demonstrated with the isolation of pure berberine from the fraction active against lung cancer cells.

In contrast to Xinxian, U.S. Pat. No. 6,290,995, the purity of one of the compositions in this invention i.e. berberine, is established using high-pressure liquid chromatography and identified unequivocally by different spectral methods, such as UV, mass spectra, 1H-NMR comparison of the isolated compound with those of published 1H-NMR spectra of authentic berberine and epiberberine and confirmed by comparison of isolated compound 13C-NMR with the published 13C-NMR spectra data of berberine. Furthermore, the invention describes a method of identifying for the first time the selectivity of pure berberine against lung cancer cells without any toxicity to other cells. As far as is known, this has not be disclosed previously.

No previous reports or data suggested that berberine is useful for selective control or treatment of human lung cancer. In fact, both Dai (1993) and Xinxian (2001) reported that their product (probably, not pure) berberine was nonselective. Furthermore, to date, no information has been published with respect to the selective activity of the salts or derivatives of berberine against lung cancer cells.

### SUMMARY OF THE INVENTION

The present invention is intended to overcome one or more deficiencies of the related art. Specifically the invention describes:
1. Compositions and methods for inhibiting cancer or viral infection comprising administering a therapeutically effective amount of:
   (a) berberine for inhibiting growth of lung cancer. Berberine was isolated in pure form from an organic extract of Coptis teeta in this instance for the study. It is to be clearly understood that although it may be obtained from Coptis teeta extract or other species of plants, preferably the pure compound is prepared by synthesis, partial synthesis or other means;
   (b) an organic extract or compounds therein of Myanmar medicinal plant, Coptis teeta, for inhibiting growth of lung cancer;
   (c) an aqueous extract or alternatively compounds therein after organic solvent extractions of Coptis teeta inhibiting growth of breast cancer;
   (d) an extract or compounds therein of Myanmar medicinal plant, Coptis teeta, with an anti-HIV activity for treating AIDS;
   (e) berberine, by itself or in combination with compounds isolated from an extract of Coptis teeta with an anti-HIV activity for treating AIDS.
2. Compositions and methods for treatment suitable for use in the therapeutic application of malaria which comprising of:
   (a) an extract of Myanmar medicinal plant, Coptis teeta, with an antimalarial activity;
   (b) berberine, by itself or in combination with compounds from an extract of Coptis teeta with an anti-malarial activity.

The active compound, berberine, is isolated by a method comprising the steps of preparing an extract from the rhizome of Coptis teeta to mediate a selective cytotoxic profile against human lung cancer in a subject panel of human cancer lines, conducting a bioassay-directed fractionation based on the profile of bioactivity using cultured human lung cells (Lu-1) as the monitor, and obtaining pure berberine therefrom as the active compound. The pure berberine can be used to prevent or inhibit tumor growth or can be converted to a derivative to prevent or inhibit tumor growth.

The invention describes the positive identification of berberine as the key active compound from Coptis teeta which is selectively active against tumor cells and specifically against lung cancer cells. The problem of insufficient availability and reproducible specifications associated with natural compounds may be addressed by utilizing synthesized or partially synthesized berberine and its derivatives or salts.

These and other aspects of the present invention will be apparent from the description of the invention disclosed below, which descriptions are intended to limit neither the spirit nor scope of the invention but are only offered as illustration of the invention.

### DESCRIPTION OF THE DRAWINGS

FIG. 1 shows a method of solvent extraction of rhizome material of Coptis teeta Wall.

FIG. 2 shows the chemical structure of berberine.

FIG. 3 shows table 1, showing the biological activity of the extracts of the rhizome material of Coptis teeta Wall.

FIG. 4 shows table 2, showing Lu I activity of column chromatographic fractions from the CHCl₃ and petroleum ether extracts of Coptis teeta Wall.

FIG. 5 shows table 3, showing the weight of column chromatographic fractions from the EtOAc extract of Coptis teeta Wall.

FIG. 6 shows table 4, showing the weight of column chromatographic fractions from fraction 5 of the EtOAc extract of Coptis teeta Wall.

FIG. 7 shows table 5, showing the cytotoxic activity of isolates from Coptis teeta Wall.

FIG. 8 shows table 6, showing the antimalarial activity of isolates from Coptis teeta Wall.

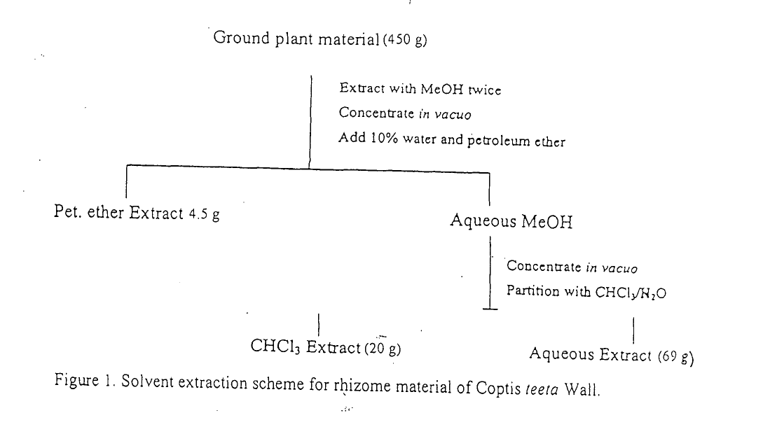

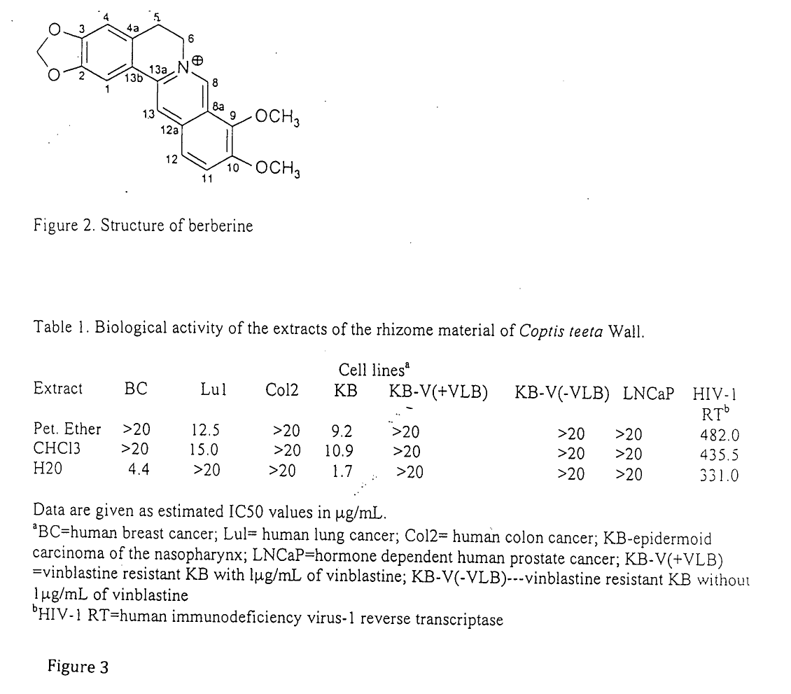

### DETAILED DESCRIPTION OF THE INVENTION

The present invention relates to berberine generally. More specifically this invention concerns berberine's selective cytotoxicity against human lung cancer cell line. Because of its known safety profile at high doses, it may be used preferentially in the treatment of lung cancer.

Furthermore compositions for inhibiting tumor/cancer or viral infection and malaria and methods of obtaining these compositions from Myanmar medicinal plant, Coptis teeta are described. These compositions may be used in combination with a potentiator or a chemotherapeutic agent. The compositions may be used for treating lung cancer, breast cancer, AIDS and malaria. Additionally, this invention relates to berberine and other components contained in this plant that are identified to be active against HIV which may be used in the treatment of AIDS. Berberine and other compounds contained in this plant with anti-malarial activity may be also used against malaria.

Search for Drugs Derived from Natural Products

Natural products (produced by plants, fungi, bacteria, protozoans, insects and animals) or their derivatives comprise of nearly one half of the top selling drugs in the world. From around 500 new drugs approved in the United States between 1983 and 1994, 30% of them were natural products or their derivatives. Also, 60 to 80% of the anti-cancer drugs and antibacterials were natural products. Nine among the twenty best selling, non-protein drugs in 1999 were obtained from or developed as a result of leads generated by natural products with combined sales of over $16 billion.

Ethno-medicine (the use of plants by man to ameliorate illness since time immemorial) plays a significant part in the new drugs discovery. Chemical studies involving the isolation of the active compounds from plants used in traditional medicine provided nearly 80% of the current 122 compounds from 94 plant species. Since drug discovery and development requires great capital ($500 million per new drug) and time (more than 10 years), the ethno-medical approach becomes quite attractive alternative, a shortcut for finding new drugs. The potential benefits provided are (a) safety proven through human use for a long time (b) the ethno-medical information collected over hundreds of years can be used as an indicator of specific biology activity suitable for particular high-throughput assays developed from knowledge of current biology.

Myanmar Traditional Medicine

The term traditional medicine is frequently used to specify a system of health care utilizing locally available medicinal plants exhibiting therapeutic effects that may contain bioactive chemical components. The goal of the research in traditional medicine is to search for indigenous plants with low toxicity profile with enhanced safety and efficacy, and as a potential source for discovering new drug compounds, preferably those that can be synthesized readily or obtained by other means easily.

Selection of Source Materials

There are two fundamental approaches to the discovery of new medicinal agents from natural sources. The first is to randomly test every available plant material for activity against array of in vitro bioassays. The success rate for this method is very low (3%).

Approximately 66% to 88% of the world's population uses plants as a primary source of health care. Consequently, an alternative approach to discovering medicinal agents from plants is to consider those plant materials which are used in the thousands of indigenous cultures worldwide for specific diseases, and which clinically have been demonstrated to be effective. This is the so-called ethno-medical approach and is responsible for about 74% of the plant-derived prescription products on the market today.

Among the many systems of plant medicines still to be evaluated, Myanmar, for a variety of different reasons, is especially well suited for plant collection at this time. The increased sophistication of modern plant selection methodology and the recent development of rapid and reliable screening programs contributes greatly to the cost-effectiveness of studying natural products as a formal part of drug discovery process.

Myanmar fits the description given by Cox in 1994 for a country and culture that would be expected to provide a high yield of medicinal plants with verifiable pharmacological properties, namely:
1. Myanmar has a long and respected history involving indigenous healers and traditional medicine;
2. Myanmar has varied geographical regions resulting in diverse flora;
3. The population has been stable, residing in the region for over several thousand years; and
4. Myanmar has extensive tropical forests especially rich in undiscovered medicinal plants.

Plant samples from Myanmar were acquired by the inventor's company, PharmChem. Inc., based on current local use for malaria, cancer or AIDS. Plants were selected through discussions with indigenous medical practitioners and local scientists. The following description of healing properties in plants, among others, were addressed as follows:

What plants are used for the treatment of cancer, sores, non healing wounds, abscesses, warts, infectious diseases, parasitic diseases, immune disorders, general weakness and loss of weight, vomiting blood, fever and respiratory problems, and diseases caused by viruses?

Plants which possessed three or more of the above indications were considered likely candidates for selection. Selected plants were further catalogued into a database using the correct nomenclature. The healing properties description is used as an important indicator to identify medicinal plants containing compounds with anti-tumor, antimalarial or anti-HIV chemical activities.

Samples were acquired under their local names and were subsequently identified by the local botanists or through comparison with herbarium samples at Chicago Field Museum of Natural History. Since its inception, Pharmchem Inc. has acquired many different plant species from Myanmar. The invention disclosed is the outcome of the results of one of the Myanmar medicinal plants.

Processing of Myanmaran Medicinal Plant Specimens (Extraction)

Extractions of plant materials, isolation and structure elucidation of compounds, were performed. Extracts, fractions and isolated compounds of plants were evaluated against three basic disease states: cancer, AIDS and malaria. The results were reviewed based on currently accepted and well known standards for evaluating biological activity for plant extracts, fractions and isolates in these assays. (Angerhofer 1992, Monks et al 1991, Tan et al 1991 and 1992, Geran et al 1972, Skehan 1990.)

The results of the extracts, fractions and isolated compounds of one of the Myanmar medicinal plants obtained from the above-described process, Coptis teeta Wall, will be the basis of this invention disclosure.

### Plant Description

The species of Coptis are small stemless and rootstock perennial herbs. Coptis teeta Wall (Family: Ranunculaceae) is a small, rhizomatous, or stoloniferous perennial medicinal herb. The local name in Myanmar is Khan-Tauk. The species is described in the literature (Pandit and Babu) as follows:

“LEAVES: Petiolate, ternatisect, glabrous or minutely hairy on prominent veins; leaflets 3, ovate-lanceolate, pinnatifid, lobes incised, terminal lobe largest. INFLORESCENCE: Scapose monochasial cyme; peduncle as long as or longer than leaves. Flowers: 1-5, pedicellate, pentamerous, actinomorphic, dichlamydeous; bracts and bracteole leafy. SEPALS: 5, oblonglanceolate, acute, prominently nerved, glabrous and twice as broad as petals, 0.5−0.6×0.15-0.25 cm. PETALS: 10, obovate-cuneate, clawed (0.20-0.25 cm long stalk), obtuse and spathulate, 0.4−0.5×0.1-0.15. STAMENS: Many, free; filament stout, 0.3-0.4 cm long; connective flattened bearing four-lobed anther. CARPELS: 9-13, stipitate, free, style recurved into beak-like structure, stigma not well-differentiated; ovules 4-6; placentation marginal. FRUIT: Follicle, laterally compressed, prominently nerved, 1.15−1.50×0.35-0.5 cm. SEEDS: 0-6, smooth, ovoid, glaborous, dark brown, 0.25−0.35×0.06-0.08 cm.

Coptis teeta Wall grows in highly specialized niches, temperate evergreen broad-leaved forests, and in a narrow range of latitudinal (27 to 29 degrees) and altitudinal (2350-3100 m) distribution. The plant contains alkaloids of the proto-berberine type. Its rhizome is the commercial source of berberine.

The plant has been regarded as an endangered species listed in Red Data Books. Hence, it is very fortunate that this plant was studied in time to verify and record its usefulness as a potential drug and utilized it as a means to identify and obtain its bioactive compounds, such as berberine, which unexpectedly provided its new utility as a lung cancer therapy, as is disclosed in this invention. Fortuitously, this compound can preferably be obtained by synthesis and other means. Besides offering commercial advantage for the production, the danger of depleting this endangered species for the production of the drug is nil, which is an added benefit. It provides more evidence for the conservation of both the plant and the knowledge of the use of the plant for mankind. It is estimated that the cost of every medicinal plant that became extinct before its use has been verified and recorded for future development is a loss of $200 million per year revenues in financial terms and another potential medicine lost to humanity.

Methods

Selection of the Myanmar Medicinal Plants for Study

Ethno medical data and other information described earlier were part of the reason, Coptis teeta was included among the Myanmar medicinal plants to be screened for anticancer, anti-HIV and antimalarial activities.

Detailed Description of the Work on the Specific Plant, Coptis Teeta Wall, that is the Subject of the Invention

The work on the Myanmar medicinal plant, Khan-tauk (Coptis teeta Wall) is described in the following section.

Plant Material

The rhizome of Coptis teeta was collected in Myanmar. The plant was identified by Kyaw Soe, a Professor Emeritus at the University of Yangon.

The Solvent Extracts of Coptis teeta Wall

The rhizome material (450 g) was ground and percolated overnight at room temperature, and then extracted twice with 100% MeOH (IL×2). The extracts were concentrated in vacuo and suspended in H₂O (10:1). The aqueous MeOH extract was then partitioned with petroleum ether (1:1) twice to separate a petroleum ether soluble extract. The residual extract was further partitioned with CHCl₃ (1:1) to separate the CHCl₃ soluble extract from the aqueous soluble extract. Each of the extracts was dried in vacuo at 35° C. and weighed to afford a petroleum ether extract (4.5 g), a CHCl₃ extract (20 g), and an aqueous extract (69 g), respectively.

A further sample of recollected rhizome material (800 g) from Myanmar was extracted using the same procedure described above, except for using EtOAc, instead of CHCl₃. Petroleum ether (8.9 g), EtOAc (12 g), and aqueous (159 g) extracts were obtained.

The Bioassays of the Solvent Extracts

The plant partitions were evaluated using in vitro bioassays based on a panel of human cancer cell lines: BC, Human Breast; Col2, Human Colon; KB, Human Carcinoma of the Nasopharynx; KBV1, Vinblastine-resistant KB; LNCaP, Hormone-dependent Human Prostate, and ZR75-1, Hormone-dependent Human Breast. These lines represent the main priorities for anticancer screening of the National Cancer Institute and have been well documented (Lin et al., J. Nat. Prod. 56: 22-29 (1993). In addition, samples were evaluated against the HIV-1 reverse transcriptase assay and, if active, against HIV-2 RT assay and a TIBO-resistant HIV-1 strain. Furthermore, some samples were tested for antimalarial activity (Angerhofer, Advances in Natural Products Chemistry, ed. Atta-ur-Rahman, pp 311-329 (1992)).

Thus, extracts, fractions and isolated compounds of plants were evaluated against three basic disease states: cancer, AIDS and malaria according to the published and established protocols and procedures. The results were reviewed based on currently accepted and well known standards for evaluating biological activity for plant extracts, fractions and isolates in these assays. ((Angerhofer, Advances in Natural Products Chemistry, ed. Atta-ur-Rahman, pp 311-329 (1992), Monks et al., J. Nat'l Cancer Inst. 83: 11, 757-766 (1991), Tan et al., J. Nat. Product 54:143-154 (1991), Tan et al., Biochem. Biophysics. Res. Commun. 185:370-378 (1992), Geran et al., Cancer Chemother. Rep., Part 3, 3: 1-17 (1972), Skehan et al., J. Nat'l Cancer Inst. 82:1107-1112 (1990).)

The CHCl₃ partition demonstrated cytotoxic activity against the Lu1 (IC50 15.0 μg/mL) and KB (10.9 μg/mL) cancer cell lines, while the aqueous extract exhibited cytotoxic activity against KB (1.7 μg/mL), and BC (4.4 μg/mL) cancer cells, and the petroleum ether extract displayed cytotoxic activity against Lu1 (IC50 12.5 μg/mL) and KB (9.2 μg/mL) cancer cells. (See FIG. 3, Table 1).

Column Chromatographic Separation of the Solvent Extracts

Chloroform and Petroleum Ether Extracts Fractionation

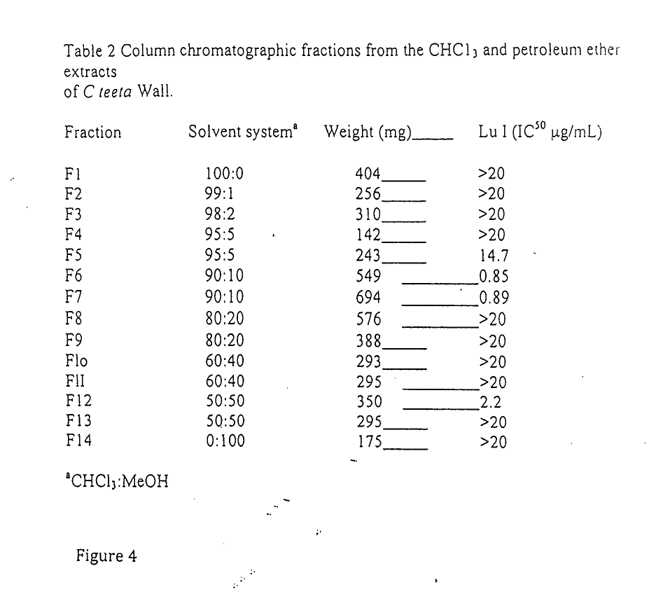

The CHCl₃ was fractionated over a silica gel column (5×60 cm, 700 g, 63-200 μm). For the column chromatography, a CHCl₃/MeOH gradient solvent system was applied with a gradual increase of the MeOH content. Fourteen fractions were collected based on the similarity of their TLC profiles. The fractions were then evaluated for their cytotoxic activity against Lu1 cancer cell line in order to determine the active fractions. The active fractions were fraction 5 (IC50 14.7 μg/ML), 6 (0.85 μg/mL), 7 (0.89 mg/mL), and 12 (2.2 μg/mL), respectively. (See FIG. 4, Table 2).

Isolation of Berberine

Berberine (1.42 g) was crystallized with MeOH as yellow needles from fractions 5, 6, 7, and 12. The yield of the alkaloid was 0.32% (w/w). The purity of the compound was established using high-pressure liquid chromatography (HPLQ monitoring at 254 nm. For the HPLC chromatography, a mixture of CH₃CN/H₂O (0.1% trifluoroacetic acid) solvent system was applied with a gradual increase of CH₃CN content from 20:80 to 100:0 in 30 minutes. The isolated compound was identified unequivocally as berberine by different spectral methods, such as UV, mass spectra, 1H-NMR comparison of the isolated compound with those of published 1H-NMR spectra of authentic berberine and epiberberine and confirmed by comparison of isolated compound ¹³C-NMR with the published ¹³C-NMR spectra data of berberine by high-pressure liquid chromatography

Isolation of Coptisine

Coptisine (157 mg) was crystallized with MeOH as yellow needles from fractions 8 and 13. The yield of the alkaloid was 0.035% (w/w).

Ethyl Acetate Extract Fractionation

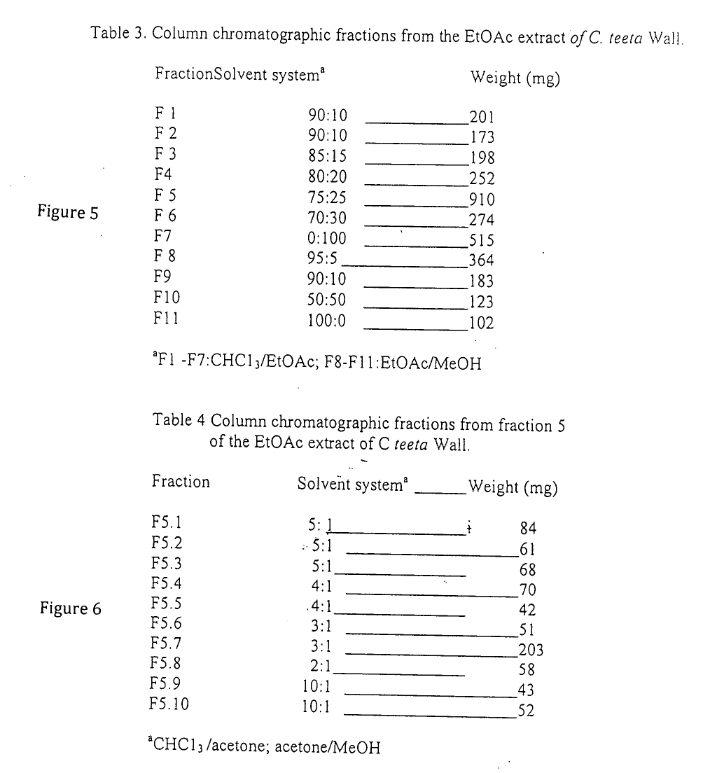

The EtOAc extract (12 g) was fractionated over a silica gel column (8×90 cm, 2,240 g). Eleven fractions were collected eluting with gradient combinations of CHCl₃/EtOAc and EtOAc/MeOH solvent systems sequentially. (See FIG. 5, Table 3).

Fraction 5 (910 mg) was further fractionated over a silica gel column (4.4×28 cm, 210 g, 40-63 μm) as the fraction displayed three major spots separated distinctly in its TLC profile. A total of ten subfractions were collected eluting with CHCl₃/acetone and acetone/MeOH solvent systems sequentially. (See FIG. 6, Table 4).

Isolation of (±)-Chilenine

(±)-Chilenine (25 mg) was isolated from subfraction F5.4 (70 mg) (See table 4) by preparative TLC using a CHCl₃/MeOH (7:1) solvent system. The yield of the alkaloid was 0.003 1% (w/w).

Acetylation of (±)-Chilenine

(±)-Chilenine (2 mg) was acetylated at room temperature with Ac20 (0.2 ml) and pyridine (0.5 ml) for 24 hrs. After removal of the reagent, the acetylated compound 10a (1 mg) was purified by preparative TLC, using a mixture of CHCl₃/MeOH (10:1).

Isolation of Noroxyhydrastinine

Noroxyhydrastinine (5 mg) was isolated from subfraction F5.6 (51 mg) by preparative TLC, using a mixture of CHCl₃/MeOH (8:1). The yield of the alkaloid was 0.0006% (w/w).

Isolation of (+)-Lariciresinol

A subfraction F5.7 (203 mg) was fractionated over a silica gel column (1.8×20 cm, 82 g, 63-200 um), using a mixture of CHCl₃/MeOH. (40:1). Four fractions were collected based on the similarity of their TLC profiles: F5.7.1 (28 mg), 5.7.2 (49 mg), 5.7.3 (34 mg), and 5.7.4 (27 mg). (+)-Lariciresinol (30 mg) was isolated from subfraction F5.7.2 (49 mg) by preparative TLC using a mixture of CHCl₃/acetone (6:1). The yield of the compound was 0.0036% (w/w).

Isolation of 3,4-dehydronoroxyhydrastinine

3,4-Dehydronoroxyhydrastinine (2 mg) was isolated from subfraction F5.8 (58 mg) by preparative TLC using a mixture of hexane/EtOAc/MeOH (6:6:1). The yield of the alkaloid was 0.00024% (w/w).

Results and Discussion

Six known compounds, berberine, coptisine, racemic chilenine, lariciresinol, noroxyhydrastinine, and 3,4-dehydronoroxyhydrastinine were isolated from the rhizome of Coptis teeta Wall. in this study.

Biological Activities of the Isolates from Coptis teeta Wall.

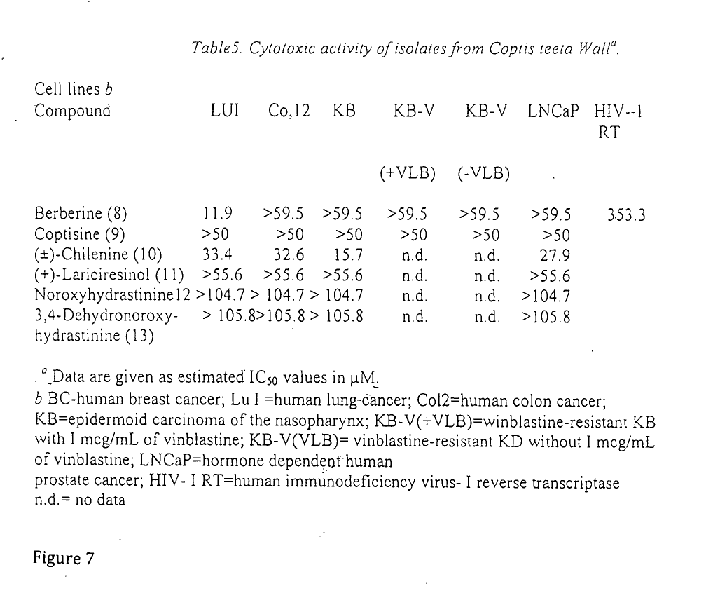

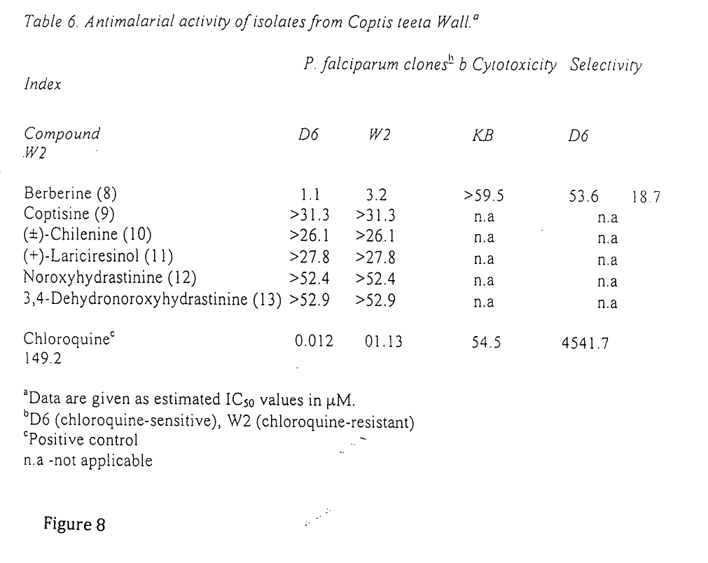

Table 5, shown in FIG. 7, lists the cytotoxic activity results for berberine, coptisine, racemic chilenine, (+)-lariciresinol, noroxyhydrastinine and 3,4-dehydronoroxyhydrastinine. Berberine exhibited selective cytotoxic activity with an IC₅₀ value of 11.9 μM against human lung cancer cell line. (±)-Chilenine showed moderate activities with IC₅₀ values of 15.7-33.4 μM against all of the human cancer cell lines tested. The remainder of the isolates did not display any significant cytotoxic activity.

The activity of berberine appeared to explain the cytotoxic activity of the initial solvent extracts and the subsequent fractions from which the compound was isolated. It is therefore concluded that berberine was one of the active principles in C. teeta Wall, which is responsible for the strong cytotoxic activity against human lung cancer cell line.

As shown in Table 5, in vitro growth of lung cancer cells was inhibited by pure berberine with an IC sub 50 value of 11.9 μM. However, none of the other cancer cell lines tested was affected by berberine (IC sub 50 values of greater than 59.5). Such clearly defined cell-type specificity demonstrated by berberine is both new and unexpected. Until this invention disclosure, no previous report on data suggested that berberine would be useful for the selective control and treatment of lung cancer. No information has been published with respect to this selective activity of berberine. Quite the contrary, it was indicated to be non selective, e.g., Dai et al 1993 and Xinxian 2001.

As shown in Table 1 (FIG. 3), all of the extracts showed strong anti-HIV activities against human immunodeficiency virus-1 reverse transcriptase (HIV-1 RT) with values of 482.0, 435.5 and 331.0 for the extracts of pet ether, chloroform and water (Tan et al. J. Nat. Prod. 54:143-154, 1991). Pure berberine is also active with a value of 353.3 (see Table 5).

All of the isolates were tested for antimalarial activity against two clones of P. falciparum. (See FIG. 7, Table 6.) Berberine showed strong activity with ICₛₒ values of 1.1 and 3.2 μM against the D6 and W2 clones of P. falciparum, respectively. These data agreed with the literature data, IC₅₀ values of 0.42 μM against the D6 and 0.44 μM against W2 clones of P. falciparum (Vennestrom and Klayman, 1988). The selectivity index (SI) of berberine was 53.6 (D6) and 18.7 (W2), whereas the SI values of the positive control, chloroquine, were 4541.7 (D6) and 149.2 (W2).

### CONCLUSION

From the rhizomes of Coptis teeta Wall, one isoindolobenzazepine alkaloid, two protoberberine alkaloids, one 7,9-epoxytetrahydrofuranoid lignan, and two simple isoquinolone alkaloids, were isolated. Two of the alkaloids, (±)-chilenine, and 3,4 dehydronoroxyhydrastinine, were isolated from the Coptis genus for the first time, while (+)-lariciresinol, and noroxyhydrastinine, were isolated for the first time from this species.

Berberine surprisingly exhibited selective cytotoxicity against human lung cancer cell line which would preferentially serve as a selective therapeutic agent for the treatment of lung cancer with no side effects. This selective cytotoxicity of berberine and its general lack of toxicity even at high doses should afford a favorable therapeutic index. Coupled with the abundant data already available on its synthesis, pharmacology, pharmacokinetics, clinical trials, etc., berberine would have a fast track in its development as a specific lung cancer remedy.
1. Berberine or its salts or derivatives are identified as the active compound for selectively inhibiting lung cancer, potentially without toxic side effect.
2. Berberine is preferably obtained by synthesis or partial synthesis or other means. But it can also be obtained from natural sources, such as Coptis teeta from Myanmar and other countries, or from other berberine containing plants.
3. Berberine and its derivatives are also active against HIV. Therefore, it may be a safe new drug for the prevention of AIDS, alone or in combination with other antiviral agents.
4. Composition and method of inhibiting tumor or viral infections and malaria without toxic side effects is disclosed.
5. A natural composition from the rhizome of Coptis teeta may be used as a safe new drug for the prevention of human lung cancer.
6. A natural composition from the rhizome of Coptis teeta may also be used as a safe new drug for the prevention of human breast cancer.

What is claimed is:

1. A method for selectively inhibiting growth of human lung cancer cells comprising administering to said lung cancer cells an effective amount of a composition consisting essentially of pure berberine, wherein said pure berberine is selectively cytotoxic to said lung cancer cells.

2. The method of claim 1 wherein said pure berberine has an IC₅₀ value against said lung cancer cells of about 11.9 μM.

3. The method of claim 11 wherein said pure berberine has an IC₅₀ value against said lung cancer cells of about 0.85 μg/ml or less.

4. The method of claim 1 wherein said pure berberine is selectively cytotoxic to in vitro lung cancer cells.

5. The method of claim 1 wherein said pure berberine is combined with a potentiator or a chemotherapeutic agent.

6. The method of claim 1 wherein said pure berberine comprises an effective amount of synthesized pure berberine or partially synthesized pure berberine.

7. The method of claim 1 wherein said pure berberine is extractable from Coptis teeta Wall.

8. The method of claim 7 wherein said pure berberine comprises a fraction isolated from an extract of rhizome tissue of Coptis teeta Wall, wherein said extract is selected from the group consisting of an aqueous extract, a methanol extract, a petroleum ether extract, a chloroform extract, and mixtures thereof.

9. The method of claim 7 wherein said pure berberine selectively inhibits growth of said lung cancer cells by 50% at a concentration of about 0.85 μg/ml or less.
